(19) United States
(12) Patent Application Publication
(10) Pub. No.: US 2022/0193440 A1
(43) Pub. Date: Jun. 23, 2022

(54) Light therapy device for treatment of the vagina and vulva

(71) Applicant: Dynamic Medical Technologies, Inc., St. Petersburg, FL (US)

(72) Inventor: Vivienne Reign, St. Petersburg, FL (US)

(21) Appl. No.: 17/654,274

(22) Filed: Mar. 10, 2022

Related U.S. Application Data
(63) Continuation-in-part of application No. 17/649,709, filed on Feb. 2, 2022.
Application No. 17/649,709 is a continuation-in-part of application No. 16/834,770, filed on Mar. 30, 2020, now abandoned.

(51) Int. Cl. A61N 5/06

(52) U.S. Cl. CPC A61N 5/0603; A61N 2005/0652; A61N 2005/0611; A61N 5/0616

(57) ABSTRACT

The light therapy device for treatment of the vagina and vulva includes internal and external sections. The internal section is for treatment of the vagina, the external section is for treatment of the vulva. A frame joins the two primary components of the device: an insertion structure that is covered in a pliable cover, and a flexible wrap. Insertion structure is for internal treatment of the vagina. These insertion structure includes a rigid or semi rigid body that supports two sets of leads. An external wrap provides light therapy to the vulva. The wrap includes a flexible LED array that allows the user to contour the wrap to the shape of her body while maintaining electrical continuity to avoid damage to the LEDs.

### CROSS-REFERENCE TO RELATED APPLICATION

This application is a continuation-in-part of U.S. patent application Ser. No. 17/649,709 filed Feb. 2, 2022, titled Penis phototherapy device, which in turn is a continuation-in-part of U.S. patent application Ser. No. 16/834,770, filed Mar. 30, 2020, titled PHOTOTHERAPEUTIC DEVICE FOR A PENIS AND A USING METHOD THEREOF.

### FIELD

This invention relates to the field of light therapy and more particularly to a device for the internal and external application of therapeutic light to the vagina and vulva.

### BACKGROUND

Light is an effective treatment for multiple disorders. It can increase blood flow, treat skin conditions, and improve overall health.

Effectiveness of light as a treatment is hampered by the difficulty in applying it to a specific area for a long period of time. Patients may not be willing, or able, to spend sufficient time with a device applied to an area to receive full therapeutic benefit.

Additionally, certain areas of the body are difficult to treat with light due to shape or location.

The vagina is particularly difficult to treat using light because it is internal.

What is needed is a device that can provide internal light therapy to the vagina while also providing external light therapy to the vulva.

### SUMMARY

The light therapy device for treatment of the vagina and vulva includes internal and external sections. The internal section is for treatment of the vagina, the external section is for treatment of the vulva.

A frame joins the two primary components of the device: an insertion structure that is enclosed by a pliable cover, and a flexible wrap.

The insertion structure is for internal treatment of the vagina. The insertion structure includes a rigid or semi rigid body supporting two sets of LEDs. The first set of LEDs is outward facing along the outer surface of the insertion structure, emitting light 360 degrees, toward the walls of the vagina. The second set of LEDs is at the tip of the insertion structure, also emitting light outward, upward into the vagina.

The insertion structure is surrounded by a flexible/pliable cover, the flexible cover preferably of a contoured shape. The flexible cover is removable to provide user access to the LEDs, and to allow the flexible cover to be cleaned without risk of moisture or cleaning products damaging the LEDs.

While the cover seeks to prevent contamination of the LEDs, if contamination does occur the user can remove the flexible cover and clean the LEDs manually.

The flexible cover is formed from a material that allows the light emitted by the LEDs to pass through to the vaginal walls.

The base of the flexible cover preferably includes a flare to avoid over-insertion. The contoured shape includes high points—apexes—and low points—valleys. The contoured shape fits the musculature of the vagina to increase user comfort The shape is also compatible with kegels—a type of pelvic floor exercise.

An external wrap provides light therapy to the vulva. The wrap includes a flexible LED array that allows the user to contour the wrap to the shape of her body while maintaining electrical continuity to avoid damage to the LEDs.

The wrap is optionally shaped as two wings, the wings able to bend toward and away from the insertion structure.

The flexible LED arrays of the wrap are divided into two wing LED arrays and a central LED array. The central LED array emits light around the vaginal opening. The two wing LED arrays are intended to be oriented along the vertical axis of the user, i.e., up toward the belly button and down between the legs.

Optional leg wraps help keep the device in place. The leg wraps form an X-pattern across the device itself and around the user's upper legs. The weight of the device is then supported by the user's legs, rather than via the internal structure within the vagina. The device can be supported and used under clothing if desired.

The LED light therapy provides multiple benefits, including disinfection, heating, and healing.

Colors of light include 680 nm red light and 405 nm-470 nm blue light.

The light therapy device for treatment of the vagina and vulva optionally includes an integrated source of vibration, such as a motor with offset weight. The vibration can support strengthening of vaginal muscles, or be used for pleasure.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a first isometric view of the light therapy device for treatment of the vagina and vulva.

FIG. 2 illustrates a back view of the light therapy device for treatment of the vagina and vulva.

FIG. 3 illustrates a side view of the light therapy device for treatment of the vagina and vulva.

FIG. 4 illustrates a side view, showing internal structure of the light therapy device for treatment of the vagina and vulva.

FIG. 5 illustrates an exploded view of the light therapy device for treatment of the vagina and vulva.

FIG. 6 illustrates an isometric exploded view of the light therapy device for treatment of the vagina and vulva.

FIG. 7 illustrates a second isometric exploded view of the light therapy device for treatment of the vagina and vulva.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

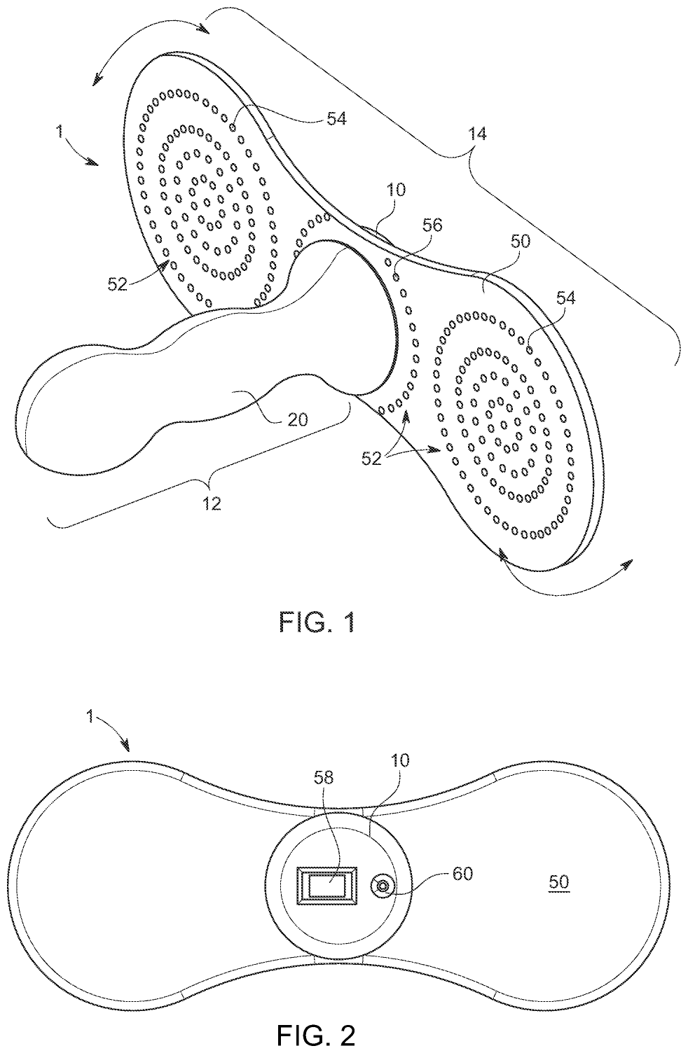

Referring to FIGS. 1 and 2, a first isometric view, and a back view, of the light therapy device for treatment of the vagina and vulva are shown.

The vaginal therapy light 1 is divided into internal treatment components 12 and external treatment components 14 connected via the frame 10.

The internal treatment components 12 include pliable cover 20 that covers an insertion structure 40 (see FIG. 4).

The external treatment components 14 include a wrap 50 that supports two flexible LED arrays 52—the wing LED arrays 54 and the central LED array 56. The wrap 50 can bend toward and away from the insertion structure 40 (see FIG. 4).

The frame 10 also includes a power switch 58 and charging port 60 connected to internal electronics (not shown).

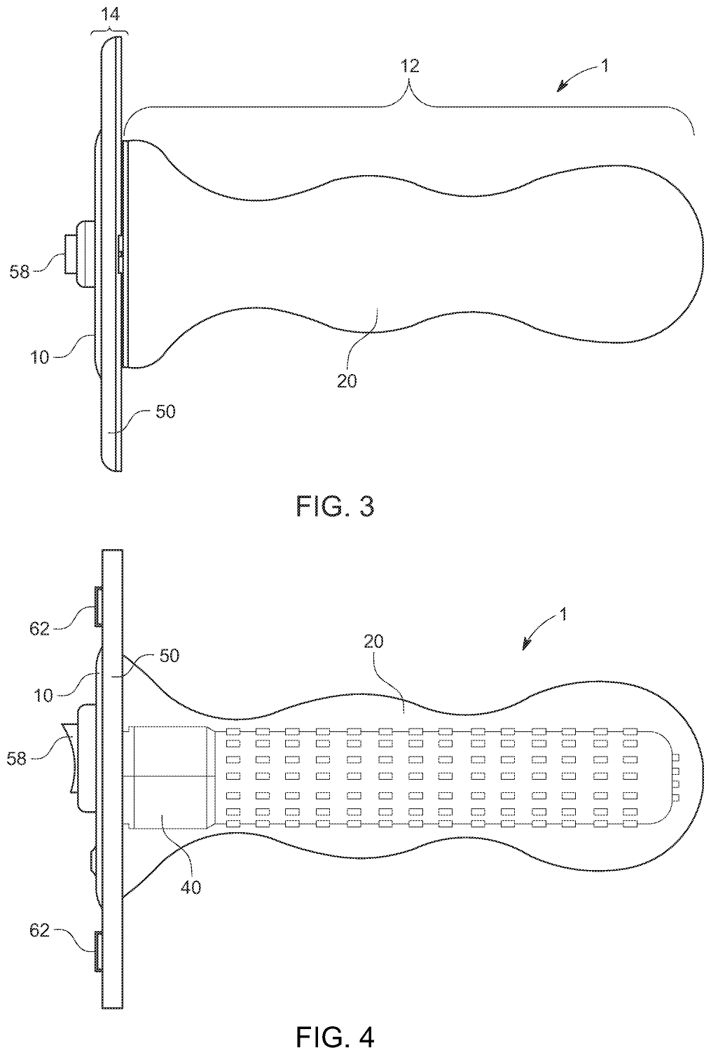

Referring to FIGS. 3 and 4, two side views, showing external and internal structure, of the light therapy device for treatment of the vagina and vulva are shown.

The internal treatment components 12 include the rigid or semi-rigid insertion structure 40 protected by the pliable cover 20.

The external treatment components 14 include the wrap 50, with optional strap slots 62 to help the user wear the device.

The frame 10 is shown with power switch 58.

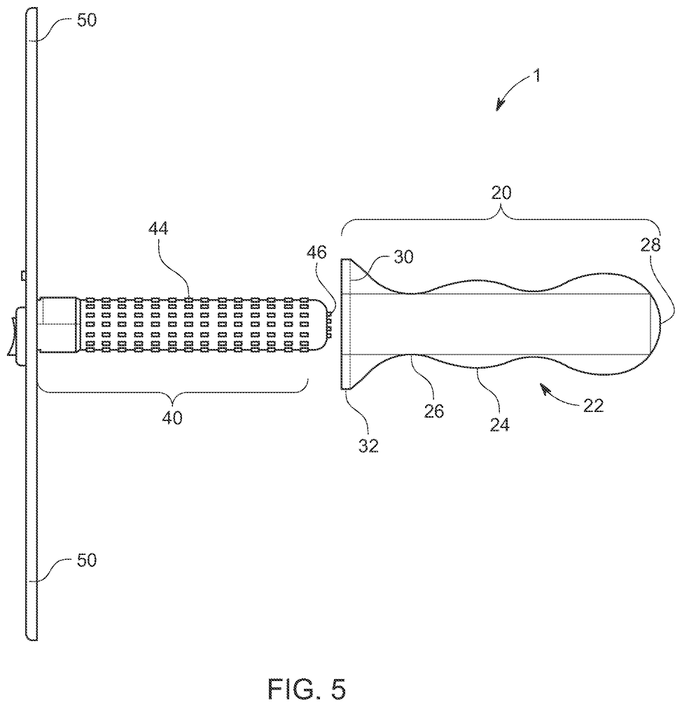

Referring to FIG. 5, an exploded view of the light therapy device for treatment of the vagina and vulva is shown.

The insertion structure 40 includes outward-facing LEDs 44 placed along its circumference, and distal LEDs 46 placed at its tip.

A pliable cover 20 is shaped for complete enclosure of the insertion structure 40, protecting the insertion structure 40 from contamination 1s and maintaining its cleanliness.

The pliable cover 20 optionally includes an external contoured shape 22. The contoured shape 22 includes high points, shown as apexes 24, low points shown as valleys 26, a tip 28, and a base 30 with optional flare 32.

Also shown is wrap 50.

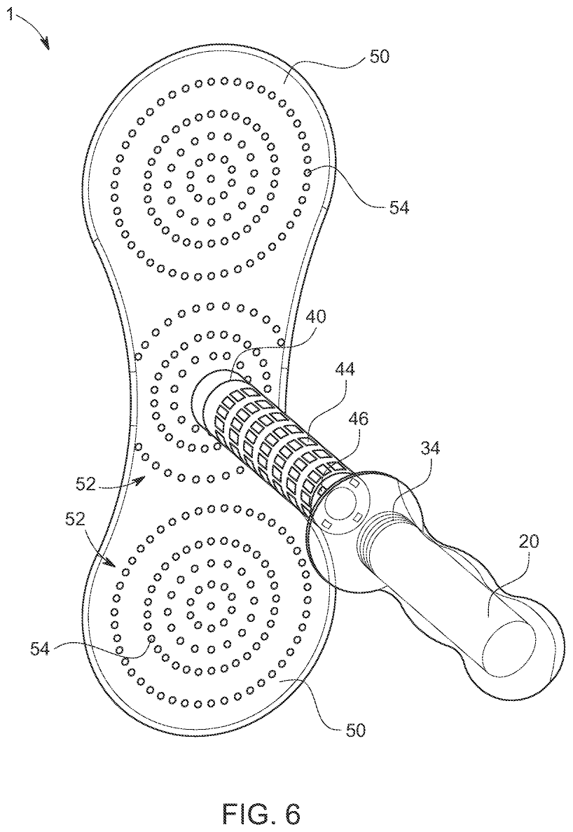

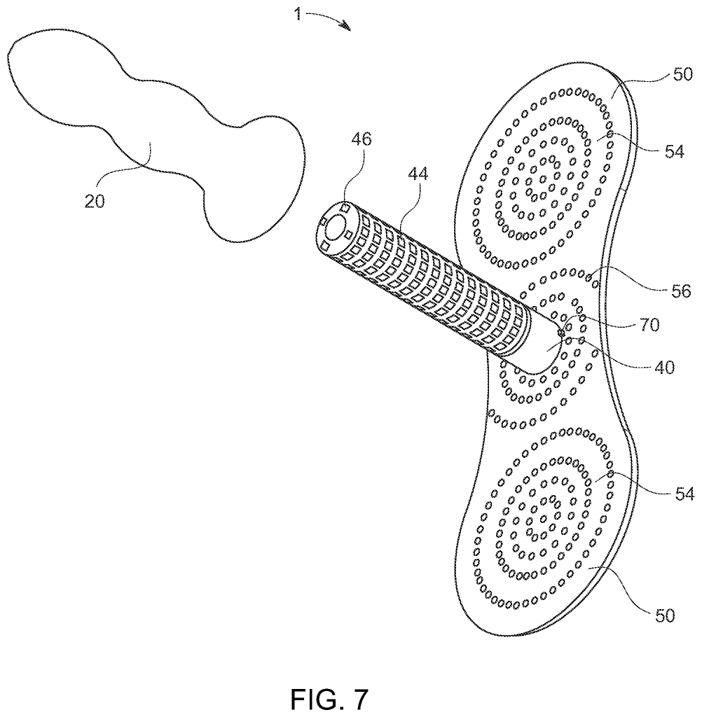

Referring to FIGS. 6 and 7, a first isometric exploded view and a second isometric exploded view of the light therapy device for treatment of the vagina and vulva are shown.

The vaginal therapy light 1 is shown with wrap 50, including the flexible LED arrays 52 formed from the wing LED array 54 and the central LED array 56.

The insertion structure 40 includes outward-facing LEDs 44 and distal LEDs 46, protected under the pliable cover 20.

Also shown are the locking ridges 34 of the pliable cover 20, interfacing with the locking teeth 70 of the insertion structure 40 to keep the pliable cover 20 in place during use.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A device for simultaneous internal and external light therapy of a vagina and a vulva of a user, the device comprising:
an internal treatment section;
the internal treatment section including an insertion structure enclosed by a pliable cover;
the insertion structure including an outward-facing LED array;
an external treatment section;
the external treatment section including a wrap;
the wrap including a flexible LED array;
whereby the user is able to simultaneously perform light therapy on the vagina and the vulva.

2. The device for simultaneous internal and external light therapy of a vagina and a vulva of a user of claim 1, wherein:
the outward-facing LED array includes a radial array of LEDs placed along a length of the insertion structure, and one or more tip mounted LEDs at an end of the insertion structure;
the radial array of LEDs and the one or more tip mounted LEDs act together to ensure complete internal light treatment of the vagina.

3. The device for simultaneous internal and external light therapy of a vagina and a vulva of a user of claim 1, wherein:
the wrap includes an upper wing and a lower wing;
the upper wing including a first wing LED array;
the lower wing including a second wing LED array;
the first wing LED array and the second wing LED array treating the vulva of the user.

4. The device for simultaneous internal and external light therapy of a vagina and a vulva of a user of claim 1, further comprising:
a central LED array;
the central LED array integrated with the wrap;
the central LED array shining light toward the vulva of the user.

5. The device for simultaneous internal and external light therapy of a vagina and a vulva of a user of claim 1, wherein:
the pliable cover has a contoured shape;
the contoured shape including areas of increased thickness, or apexes, and areas of decreased thickness, or valleys;
whereby the contoured shape increases user comfort.

6. A device for a user and their body that joins an insertable light therapy device for internal therapy with an external flexible light therapy device for external therapy, the device comprising:
an insertion structure and a wrap joined by a frame;
the insertion structure for internal therapy;
the insertion structure including a plurality of outward-facing LED light emitters;
the insertion structure including a pliable cover for comfort and hygienic protection;
the wrap for external therapy;
the wrap being flexible, allowing the user to shape the wrap to match contours of their body;
the wrap including an embedded flexible LED array for treatment of an area surrounding a point of insertion.

7. The device for a user and their body that joins an insertable light therapy device for internal therapy with an external flexible light therapy device for external therapy of claim 6, wherein:
the plurality of outward-facing LED light emitters includes a radial array of LEDs placed along a length of the insertion structure, and one or more tip mounted LEDs at an end of the insertion structure;
the radial array of LEDs and the one or more tip mounted LEDs act together to ensure complete internal light treatment.

8. The device for a user and their body that joins an insertable light therapy device for internal therapy with an external flexible light therapy device for external therapy of claim 6, wherein:
the wrap for external therapy includes an upper wing and a lower wing;
the upper wing including a first wing LED array;
the lower wing including a second wing LED array;
the first wing LED array and the second wing LED array externally treat the user.

9. The device for a user and their body that joins an insertable light therapy device for internal therapy with an external flexible light therapy device for external therapy of claim 6, further comprising:
a central LED array;
the central LED array integrated with the wrap for external therapy;
the central LED array shining light toward the user.

10. The device for a user and their body that joins an insertable light therapy device for internal therapy with an external flexible light therapy device for external therapy of claim 6, wherein:
the pliable cover has a contoured shape;
the contoured shape including areas of increased thickness, or apexes, and areas of decreased thickness, or valleys;
whereby the contoured shape increases user comfort.

11. A device for simultaneous internal and external light therapy of a user, the device comprising:
an internal treatment element;
the internal treatment element including a rigid insertion structure surrounded by a pliable cover;
the rigid insertion structure primarily rod-like in shape with an external surface and a tip;
the external surface supporting an outward facing LED array;
the tip supporting one or more LEDs;
the pliable cover enclosing the rigid insertion structure;
an external treatment element;
the external treatment element including a two-part flexible wrap with integrated LED array;
the external treatment element for application by contouring to a shape of the user;
whereby light therapy is applied both internally and externally to the user.

12. The device for simultaneous internal and external light therapy of a user of claim 11, wherein:
the outward-facing LED array includes a radial array of LEDs placed along a length of the insertion structure, and one or more tip mounted LEDs at an end of the insertion structure;
the radial array of LEDs and the one or more tip mounted LEDs act together to ensure complete internal light treatment.

13. The device for simultaneous internal and external light therapy of a user of claim 11, wherein:
the wrap includes an upper wing and a lower wing;
the upper wing including a first wing LED array;
the lower wing including a second wing LED array;
the first wing LED array and the second wing LED array act together to ensure complete internal light treatment.

14. The device for simultaneous internal and external light therapy of a user of claim 11, further comprising:
a central LED array;
the central LED array integrated with the wrap;
the central LED array shining light toward the user.

15. The device for simultaneous internal and external light therapy of a user of claim 11, wherein:
the pliable cover has a contoured shape;
the contoured shape including areas of increased thickness, or apexes, and areas of decreased thickness, or valleys;
whereby the contoured shape increases user comfort.
